(19) United States
(12) Patent Application Publication
(10) Pub. No.: US 2004/0038414 A1
(43) Pub. Date: Feb. 26, 2004

(54) Method for the pyrolysis of mercuric chloride for the subsquent analysis of the mercury

(76) Inventors: Wilfried M. Winkler, Oberbiberg (DE); Wolfgang Berkhahn, Uberlingen (DE); Christoph Schrader, Eggenstein (DE); Rudolf Paur Hanns, Karlsruhe (DE)

Correspondence Address: Robert W Becker & Associates, Suite B, 707 Highway 66 East, Tijeras, NM, 87059, US

(21) Appl. No.: 10/380,606

(22) PCT Filed: Sep. 12, 2001
(86) PCT No.: PCT/EP01/10535

(30) Foreign Application Priority Data
Sep. 13, 2000 (DE) 100 45 212.4

(51) Int. Cl. G01N 33/20

(52) U.S. Cl. 436/73

(57) ABSTRACT

The invention relates to a method for the pyrolysis of mercurie chloride in gases containing HgCl₂, Hg(0) and HCl, whereby HgCl₂ is subjected to pyrolysis in the presence of nickel as a catalyst.

The invention relates to a method for the determination or analysis of mercury. In particular, the invention relates to the analysis of mercury in mercury-containing flue gases, e.g. in exhaust gases of refuse incinerators.

The exhaust gases of refuse incinerators frequently contain mercury in the form of HgCl₂ and Hg(0), whereby these exhaust gases generally also contain considerable HCl concentrations.

Since strict limits or standards are prescribed for the emission of mercury, it is necessary to continuously monitor the exhaust gases. For this purpose, mercury analyzers are available in which elementary mercury [Hg(0)] is measured spectrophotometrically. For this purpose, it is necessary to convert the HgCl₂ contained in the exhaust gases into Hg(0).

However, within the framework of earlier work that led to the present invention, it was discovered that for a pyrolysis of HgCl₂ to Hg(0) pursuant to the following reaction equation (1), a temperature of about 900° C. is necessary. However, already starting at 800° C. there occurs a reverse reaction of Hg(0) with HCl and O₂ to HgCl₂ and H₂O (reaction equation (2)).

HgCl₂⇄Hg(0)+Cl₂ (1)

Hg(0)+2 HCl+½ O₂⇄HgCl₂+H₂O (2)

From the reaction equation (2) it can be seen that the pyrolysis temperature t should at the most be 800° C.

It is an object of the present invention to provide a method for the analysis of mercury, whereby the preceding pyrolysis occurs at temperatures of ≦800° C.

It was then discovered pursuant to the invention that elementary nickel was suitable as a catalyst material for the pyrolysis of HgCl₂ to Hg (0). In this connection, particularly suitable are nickel alloys having a nickel content of about 50 to 90%, preferably 60 to 85%, and in particular 70 to 80%. A preferred example for such an alloy is Inkonel 600 having the German industrial material code 2.4816. This steel is resistant to acid and high temperature. It contains 15% Cr, 75% Ni and 8% Fe.

The catalyst is preferably in the form of sintered material that at the same time acts as a flue gas filter. Another possibility is the use of packing material that is coated with such a nickel material. Also usable are fine wire screens.

The invention will be explained in greater detail subsequently with the aid of graphs.

Shown are:

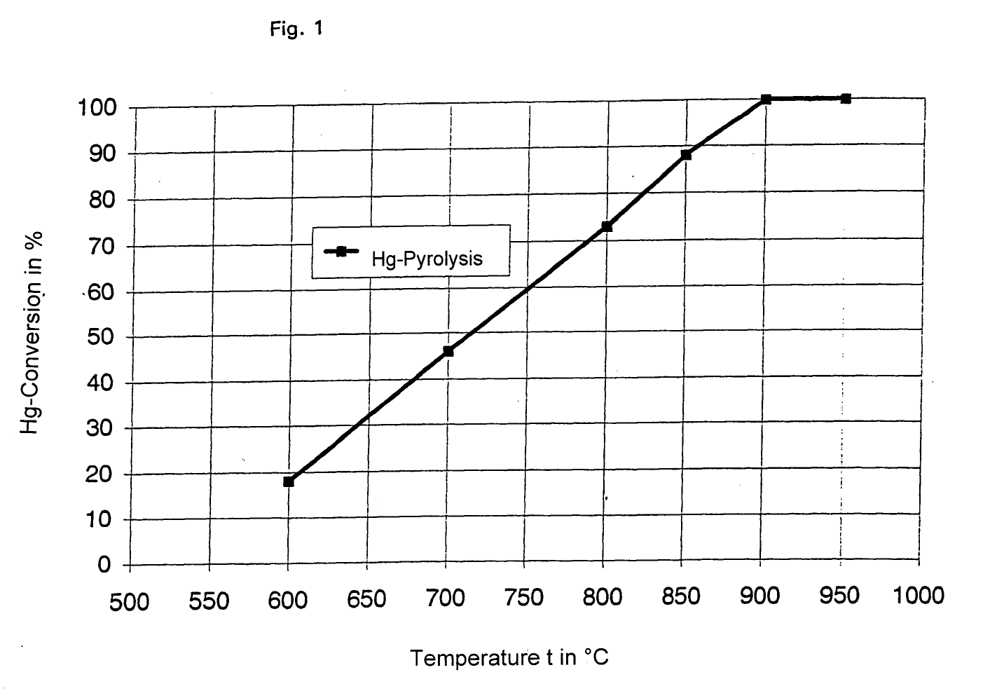

FIG. 1 a graph to explain the pyrolysis of HgCl₂ as a function of temperature;

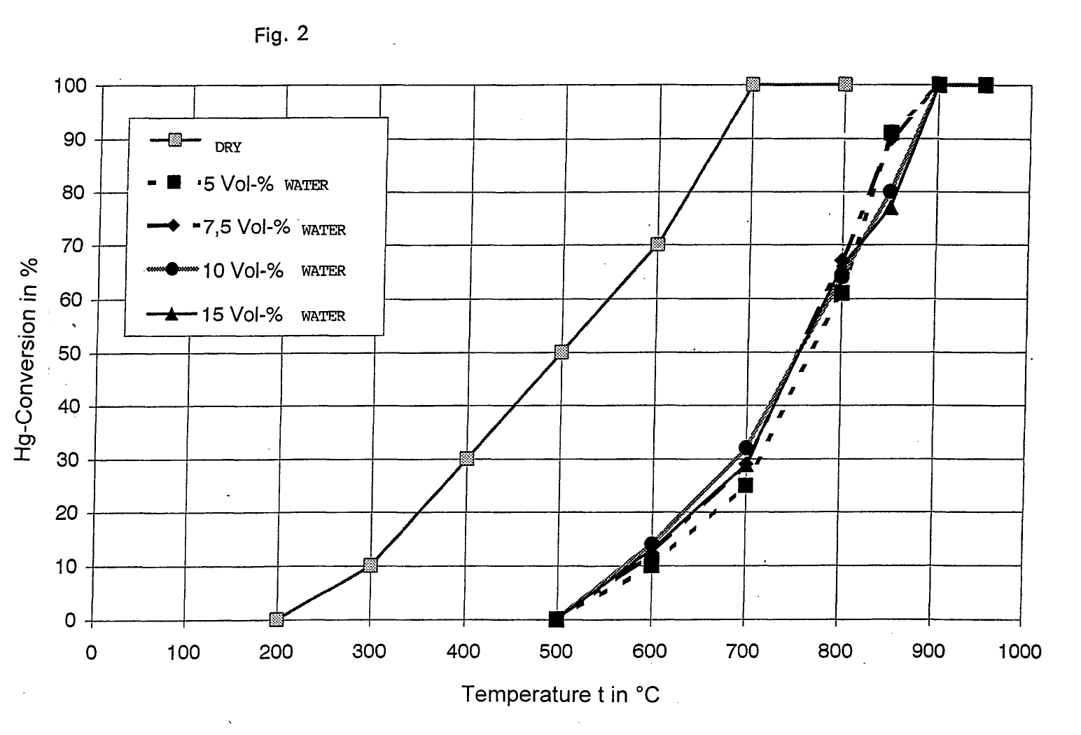

FIG. 2 the influence of moisture upon the pyrolysis of HgCl₂;

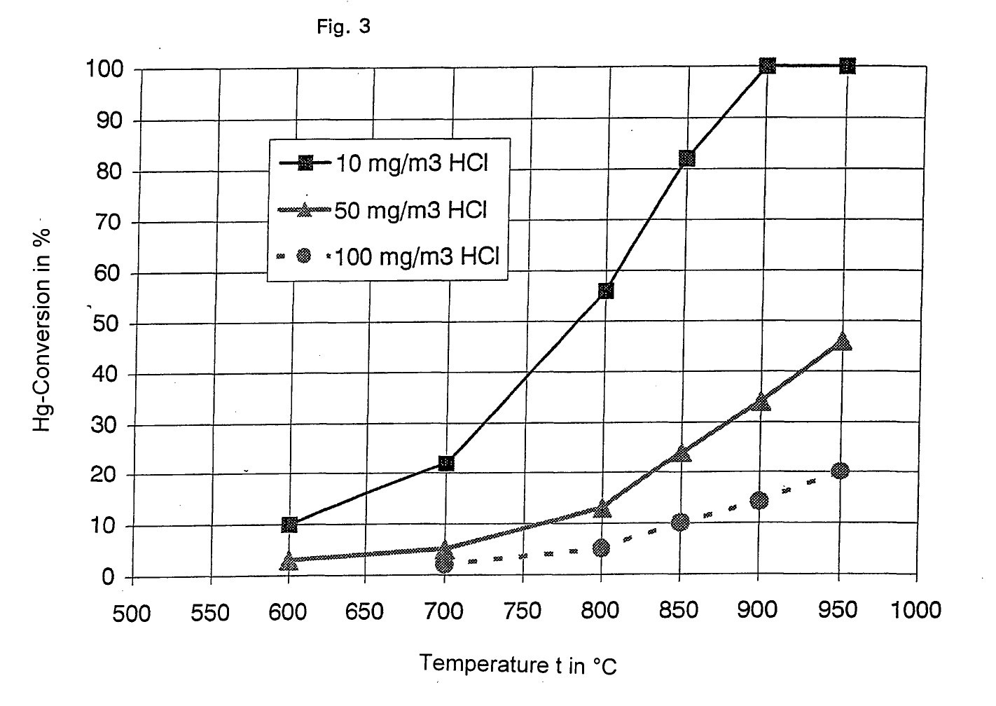

FIG. 3 the influence of HCl concentration upon the pyrolysis of HgCl₂; and

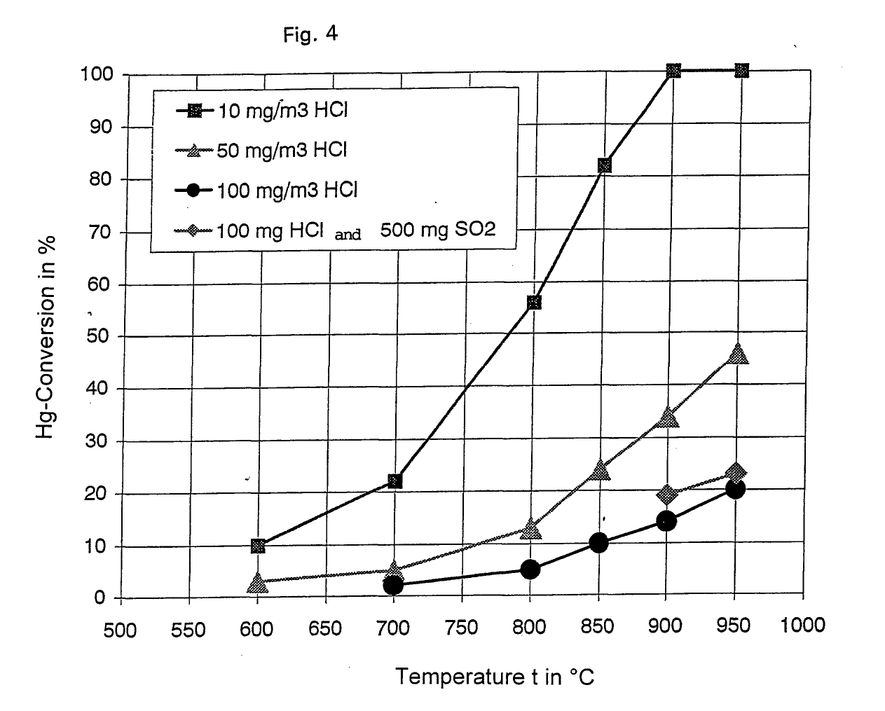

FIG. 4 the influence of SO₂ upon the pyrolysis of HgCl₂.

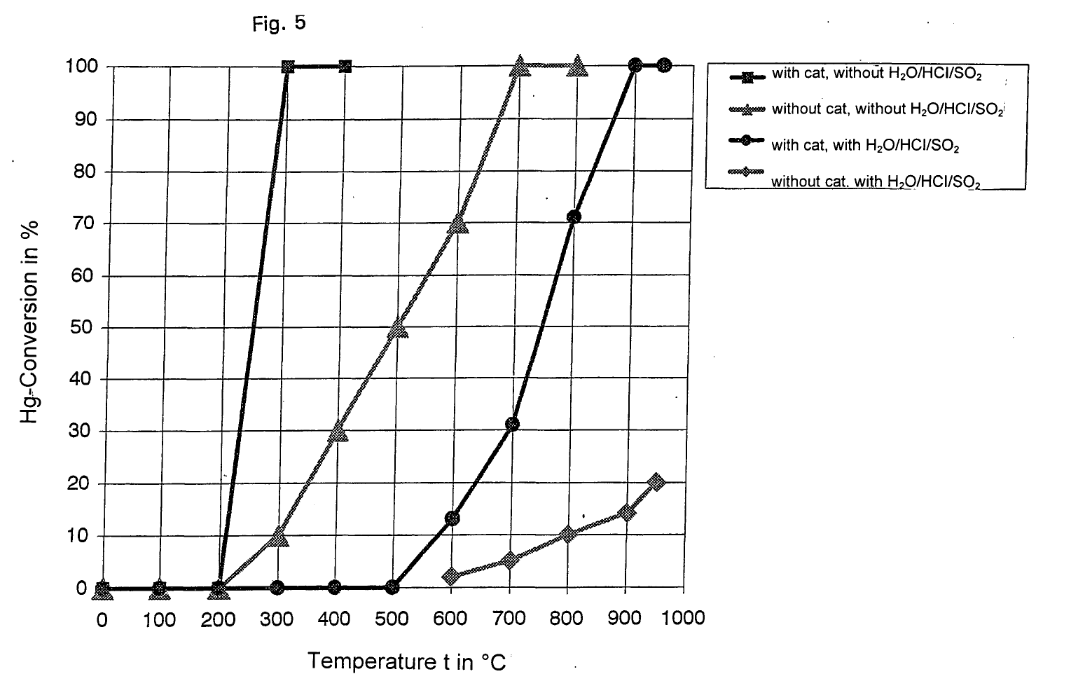

FIG. 5 the result of pyrolysis testing in the presence and absence of Ni catalyst.

FIG. 1 shows at which temperature the complete pyrolysis of HgCl₂ to Hg(0) occurs. In this connection, nitrogen having 8% by volume O₂ and 15% by volume H₂O was used as a carrier gas for all tests. The volume stream was 150 liters/h and the mercury concentration was set to about 100 μg/m³. As can be seen from FIG. 1, the pyrolysis starts at about 600° C., and is complete at an incinerator temperature of 900° C.

FIG. 2 shows the influence of water vapor or steam upon the pyrolysis of HgCl₂. The dependency of the pyrolysis upon the temperature was determined for four different water vapor concentrations between 5 and 15% by volume, as well as in the absence of water vapor. It can be seen that in general the moisture has a very great influence upon the pyrolysis. The temperature required for complete pyrolysis is shifted from about 700 to 900° C. by the presence of water vapor. In the tested range with a water vapor content of 5 to 15% by volume, no influence of the moisture content upon the pyrolysis could be observed.

FIG. 3 illustrates the influence of various HCl concentrations between 10 and 100 mg/m³ upon the pyrolysis. In this connection, HgCl₂ was added to the carrier gas in order to simulate real conditions, such as exist in refuse incinerators. At HCl concentrations of 50 and 100 mg/m³, no complete pyrolysis could be achieved; in contrast thereto, at an HCl concentration of 10 mg/m³, a complete pyrolysis was still possible at a temperature of 900° C.

FIG. 4 shows the influence of SO₂ upon the pyrolysis of HgCl₂. In this connection, 100 mg/m³HCl and 1500 mg/m³SO₂ were mixed with the carrier gas (having an Hg²⁺ concentration of 100 μg/m³. As can be seen from FIG. 4, at an incinerator temperature of 950° C. approximately the same Hg(0) concentration was achieved after the pyrolysis with and without SO₂. From this one can conclude that SO₂, which under practical conditions is contained in such flue gases, has no influence upon the pyrolysis.

FIG. 5 shows the results of pyrolysis tests with and without Ni catalyst. In this connection, nickel-plated packing material serves as the catalyst. At an Hg²⁺ concentration of 100 μg/m³, tests were carried out without H₂O, HCl and SO₂ and with 8% by volume H₂O, 100 mg/M³ HCl and 1500 mg/M³ SO₂. Without H₂O, HCl and SO₂, the temperature for the complete pyrolysis was lowered from 700 to 300° C. in the presence of catalyst. With H₂O, 100 mg/m³ HCl and 1500 mg/m³ SO₂, a complete pyrolysis could be achieved in the presence of catalyst at these conditions at 900° C., whereas without catalyst the Hg conversion was only 15%. At HCl concentrations below 100 mg/m³, the temperature for the complete pyrolysis was correspondingly lower, so that the desired temperature of 800° C. can be achieved. It follows that by the use of catalyst at lower temperatures a higher Hg conversion can be achieved.

1. Method for the analysis of mercury in gases containing HgCl₂, Hg (0) and HCl, characterized in that one carries out a pyrolysis of HgCl₂ in the presence of nickel as a catalyst.

2. Method according to claim 1, characterized in that one utilizes as a nickel catalyst a nickel alloy having a nickel content of 50 to 90%.

3. Method according to claim 1, characterized in that one uses as a nickel catalyst Inkonel (Industrial Material Code 2.4816).

4. Use of nickel during the pyrolysis of mercury compounds in the presence of HCl.

5. Use according to claim 4, characterized in that the nickel material is present as sintered material.
